(19) United States
(12) Patent Application Publication
(10) Pub. No.: US 2021/0174503 A1
(43) Pub. Date: Jun. 10, 2021

(54) METHOD, SYSTEM AND STORAGE MEDIUM WITH A PROGRAM FOR THE AUTOMATIC ANALYSIS OF MEDICAL IMAGE DATA

(71) Applicant: Raylytic GmbH, Leipzig (DE)

(72) Inventor: Frank Thilo TRAUTWEIN, Filderstadt (DE)

(73) Assignee: Raylytic GmbH, Leipzig (DE)

(21) Appl. No.: 17/114,454

(22) Filed: Dec. 7, 2020

Related U.S. Application Data
(60) Provisional application No. 62/944,583, filed on Dec. 6, 2019.

(51) Int. Cl. G06T 7/00; G16H 30/40; G16H 15/00; G16H 50/70; G16H 50/20; G16H 30/20; G06F 16/583

(52) U.S. Cl. CPC G06T 7/0012; G16H 30/40; G16H 15/00; G16H 50/70; A61B 34/10; G16H 30/20; G06F 16/583; G06T 2207/20004; G06T 2207/10116; G16H 50/20

(57) ABSTRACT

A system and method that provide the automatic extraction and processing of data from medical images from an image data archive. The processing including generating a metadata for an image, selecting an algorithm for image data analysis based on the metadata generated for the image, properties for at least two possible algorithms, and a specification of a specific image analysis to be performed, analyzing the image data with the algorithm selected from the at least two possible algorithms to produce results information, and linking the results information of image analysis and the metadata with referenceable anatomical structures of a human being or an animal within the image; and displaying the results linked to the anatomical structure.

### BACKGROUND OF THE INVENTION

This nonprovisional application claims priority to U.S. Provisional Application No. 62/944,583, which was filed on Dec. 6, 2019, and is herein incorporated by reference.

### FIELD OF THE INVENTION

The present invention relates to a method implemented in software and a device or system consisting of a computer and program code for the semi- or fully automatic analysis of medical image data, such as obtained by X-ray, CT, MR, PET or ultrasound and which are typically available in large numbers in an image archive. The field of application is the structuring or extraction of information contained in image data and thus the utilization of radiological data for research, diagnosis, and treatment of patients without or with minimal user interaction.

### DESCRIPTION OF THE BACKGROUND ART

For the diagnosis, treatment decision, follow-up of a treatment or the planning of surgical interventions, the exact knowledge of geometric dimensions, distances, angles, areas or volumes of organs, vessels, bony structures, or the pathological changes of these structures (e.g. by a tumor) is often necessary. These results are usually obtained by viewing the medical images on a screen and superimposing points, lines, distances, angles or marking areas or volumes with a digital ruler, brush, or similar digital tools. Then the visualized results are printed as a report or put in a clinical trials management system or database. To do this for a series of images, the physician or user must select the image data for each individual patient by name or other feature, view the image data associated with that patient, and select one or more images based on the acquisition date, modality, projection direction, and/or image content, depending on the purpose of the data analysis.

The entire process, from the selection of image data to the analysis and documentation of measurement results, is time-consuming, subject to variability attributed to the operator's ability to concentrate, the daily performance, individual training, underlying definitions, and user input error.

An improvement has been achieved by using a computer system with a program executed by a microprocessor, containing algorithms which, for example, use certain grey values within the image data to at least partially automate the marking of relevant pixels or voxels. Since about 2005, algorithms from the field of machine learning (generally: “Al algorithms”) can be trained to recognize characteristic image contents and to mark them. Galbuserea et al. (2016, Artificial neural networks for the recognition of vertebral landmarks in the lumbar spine, in: Computer Methods in Biomechanics and Biomedical Engineering: Imaging & Visualization 1-6.) was able to show, for example, that algorithms from the field of Convolutional Neural Networks (CNNs) can be trained to recognize edges and corner points of vertebral bodies. Korez et al. (2016, A multi-center milestone study of clinical vertebral CT segmentation, in: Computerized Medical Imaging and Graphics 49, 16-28.) was able to show that it is possible to discriminate voxels related to bony or surrounding tissue with the help of CNNs and, thus, to automatically segment 3D image data.

The placement of landmarks and image segmentation are examples for automating the image analysis process. But still, an operator is required to select from a database (e.g. a PACS or RIS system) the image data to be analyzed (e.g. regarding modality, image acquisition direction, reconstruction plane, the position or posture of the patient and the image area, the image quality and other information). The operator must assess whether these properties are suitable for a given analysis task and select the suitable image for a given task and selecting the image regions (organs, bony structures) and their physiological position and name. These are very tedious tasks.

While algorithms for the analysis of image content have been developed, a system that independently selects suitable image analyses based on image content, metadata or other data related to the patient, diagnosis or (possible) treatment, and then applies one or several algorithms to the images to extract and utilize data is needed.

A sample of related attempts to automate certain features of clinical information processing are provided below:

WO2006039358, KEAVENY et al. (2006): Generation of one model for the simulation of biomechanical loads and surgical interventions on the basis of image data and a database with reference values or ranges of values of structure-function characteristics.

WO2011021181, HAY et al (2010): Generation of one geometric model of the spine (curvature) based on reference data and imaging data and comparison of the model data with the reference data to determine pathological changes.

EP1657681, DEWAELE (2006): The sequence of several algorithms for the determination of the Ferguson angle at the spinal column is analyzed in one image by registration and calculation of the transformation matrix.

U.S. Pat. No. 8,724,865, HIPP et al. (2009): Determination of the range of motion of the spine for one image pair by registration and calculation of the translation matrix using at least two landmarks.

WO03045219, GIGER et al. (2003): Automatic determination of bone fracture risk by determining bone density at image sections one image.

US2009082637, GALPERIN (2009): Determination or characterization of a disease by combining data obtained from multiple radiological images or other data from one patient.

US2009161939, WU et al (2009): Extraction of bone characteristics using contrast agents and differential image analysis from one or more projection directions of one patient.

U.S. Pat. No. 10,255,997, CALHOUN et al. (2019): A medical analysis system based on projecting one 3D dataset on different planes and using an algorithm to color-mark malignant areas.

U.S. Pat. No. 7,046,830, GERARD et al. (2006): Method and system for the extraction of geometric data from the acquisition of one spinal column using splines whose support points are iteratively adapted to the image via a cost function.

U.S. Pat. No. 10,043,111, AKAHORI et al. (2018): Apparatus, method and program for image processing by first determining the midline of a spinal column in an image and then detecting vertebrae and intervertebral disc regions in subsequent steps by periodically evaluating the intensity differences along the midline.

US2017252107, TURNER et al. (2017): System and method for correction planning in spinal surgery. The system therefore simulates the effect of correction forces on the curvature of a spinal column on the basis of imaging and a simulation model.

U.S. Pat. No. 9,241,634, WANG (2016): Method for anatomical indexing of one patient based on 3D data of the spine.

EP1631931, WICKER (2004): Procedures and systems for image-guided implant placement.

EP3120797, NADDEO (2016): Method for identifying the optimal direction and maximum diameter of a pedicle screw.

US2010121178, KRISHNAN et al (2012): System and method for automatic diagnosis and decision support for breast imaging of one patient.

US2009046905, LANGE et al. (2009): Diagnostic support system for cervical cancer in one patient by registering various calibrated imaging data sets.

U.S. Pat. No. 7,305,111, ARIMURA et al. (2007): Automatic detection of lung cancer in low-dose CT images for one patient

U.S. Pat. No. 10,413,236, AOYAGI et al. (2019): Apparatus for automated processing of medical images by detecting a pathologically altered target area and correlating its extent with the surrounding tissue of the affected organ. This relates to individual patient errors and the disclosure suggests looking at several data sets of one patient to improve diagnostic accuracy.

EP3012759, SEEL (2019): Procedure for planning, monitoring, surveillance and/or final control of a surgical procedure in which the user manually selects different images of one patient for preoperative planning.

A system which fully automates the metadata analysis, image processor selection, and image analysis, however, would lead to a considerable relief of the clinical staff and make the immense amount of radiological data easily accessible for statistical big data analysis. Such a system would furthermore allow to select retrospective control groups for any given medical question relating to image data.

### SUMMARY OF THE INVENTION

It is therefore an object of the present invention to fully automate the analysis of a large amount of medical image data, such as is generated in clinics and radiological practices. The system or method may be implemented in an analysis device, including a computer system with connectivity to at least an image archive.

The invention may relate to a system and a method for the automatic extraction and processing of data from medical images by computer instructions executed on one or more processors and at least one interface to an image data archive including: generating a metadata for an image; selecting an algorithm for image data analysis based on the metadata generated for the image, properties for at least two possible algorithms, and a specification of a specific image analysis to be performed; analyzing the image data with the algorithm selected from the at least two possible algorithms to produce results information; linking the results information of image analysis and the metadata with referenceable anatomical structures of a human being or an animal within the image; and displaying the results linked to the anatomical structure.

The image may be filtered by matching the metadata against filter criteria. The results information may be evaluated by comparison with value ranges or thresholds indicative of the confidence of the correctness. The medical images may be enriched with embedded metadata derived by analysis of contents of the medical images with the aid of artificial intelligence algorithms. The medical images are enriched with metadata from other data sources. During generating the metadata, image-based textual data is analyzed and standardized through optical character recognition and natural language processing algorithms and output as the metadata. During generating the metadata, different sources of extracted metadata are jointly analyzed for a final prediction output as the metadata, the metadata being structured in a predefined terminology for subsequent processing.

During the selecting of the algorithm for image analysis, the selection is based on a stored association between an analysis task and the algorithm. During the selecting of the algorithm for image analysis, the selection is based on a stored association between an analysis task and the metadata of the image. The at least two possible algorithms may include: Al algorithms, computer vision, segmentation, registration, trigonometry, vector algebra, optimization functions, and/or digitally reconstructed radiographic image projection (DRR). The results information of the algorithm are associated with a metric suitable for assessing a correctness of the results information. The results information are referenced to structures of an animal or human being within the image. The permissible range of values for the results information may be limited.

The system and method uses a combination of analysis algorithms and pre-determined, stored information regarding the anatomical structure of a living subject and for the parameterization and selection of suitable algorithms and analysis sequences for a given analysis task.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a system diagram according to an implementation of the invention;

FIG. 2 is a system diagram of a metadata analyzer according to an implementation of the invention;

FIG. 3 is a system diagram of a hardware configuration according to an implementation of the invention;

FIG. 4 is a system diagram of an analysis device according to an implementation of the invention;

FIG. 5 is a processed image of a spinal segment according to an implementation of the invention;

FIG. 6 is a process for analyzing metadata according to an implementation of the invention;

FIG. 7 is a process for training a machine learning model according to an implementation of the invention; and

FIG. 8 is a process for automating analysis of clinical data according to an implementation of the invention.

### DETAILED DESCRIPTION OF THE DRAWINGS

The automation processes and the other processing and recording functions or capabilities disclosed herein may be performed by one or more computer systems. A computer system itself may include memory (RAM), one or more microprocessors with one or more processing cores, an input/output (I/O) interface suitable to access external systems (e.g. Ethernet). The computer system may also include a non-transitory storage medium (e.g. a hard drive or solid state drive) for storing and/or accessing the computer program code and computer program instructions (i.e. software). The computer system may be formed of one or more processing servers, one or more database servers, and/or one or more user terminals. The one or more processing servers and the one or more database servers may be hardware instances of cloud computing resources.

The computer program instructions may be executed by one or more processors (e.g. central processing units, graphics processing units) of the computer system. The computer program instructions may form applications or software applications which are executed to perform one or more processes. The computer program instructions may form input/output interfaces, host application programming interfaces (APIs), client APIs, data structures, or filters. The computer program instructions may be stored in a high-level language (e.g. JAVA or assembly language) and executed in binary or interpreted by one or more applications being executed on the processor.

The computer system may be distributed over several computers or servers for distributing computing load, facilitating parallel execution, adapting the hardware architecture and resources to the computing needs and for ensuring high system availability. The distribution of computers may be carried out in order to protect personal data of patients by localizing data. That is some computer systems processing patient data with personal information may be co-located at a clinical site or accessible only within the hospital intranet (“private computers”), whereas other computers are accessible to a broader group (e.g. through the internet (“public computers”)). Public computers may only be able to provide filter criteria or the definition of analysis tasks, which will be forwarded to the private computers and may not control the analysis tasks or receive the result data. User terminals may be public computers or private computers which authorize and connect with the computer system. User terminals may include memory, processor(s), hard drives, and displays for graphical depiction of results data and intermediate data.

The terms “selector”, “analyzer”, “interface”, “validator”, “generator”, “predictor”, “model”, “definition”, “component”, “algorithm”, and “device” as used herein may be implemented as hardware or software, or a combination of hardware and software. If implemented in hardware, these elements may encompass application specific integrated circuits (ASICs), field programmable gate arrays (FPGAs), integrated circuits, or other processors adapted to perform various functions. If implemented in software, the elements may include computer program instructions that when executed by a processor of the computing system cause the computing system to perform various functions. If implemented as a combination, the applications or computer instructions may be separately executed on processors and connected by hardware interfaces or shared memory.

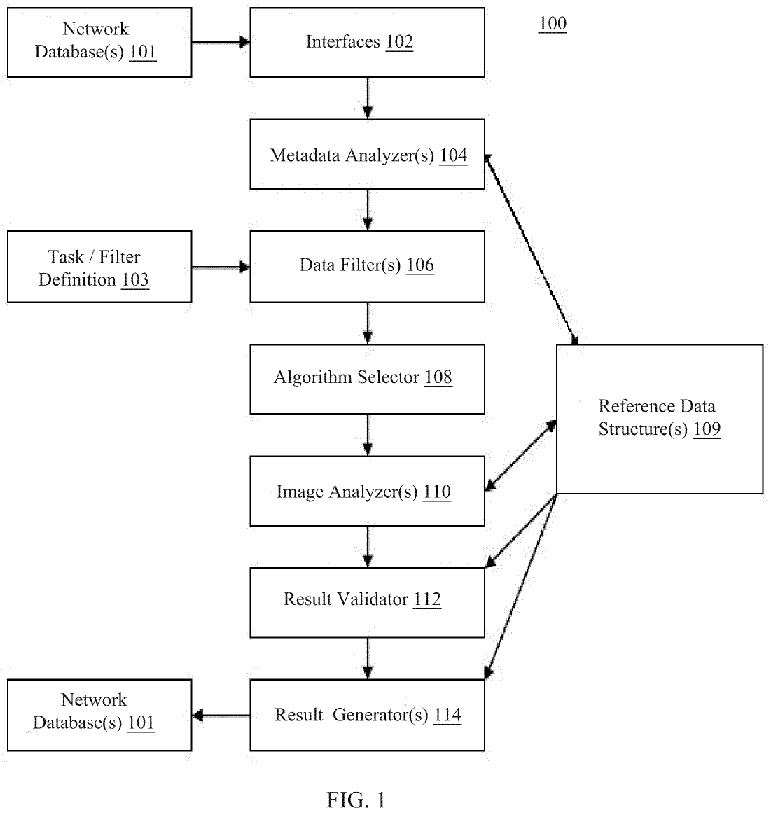

FIG. 1 illustrates an implementation of components performing an automated analysis of image data in a clinical setting. The interfaces 102 may receive image data from the network databases 101 which are made accessible to the computer system 100 and/or the interfaces 102. The network databases may include a picture archiving and communication system (PACS) or radiology information systems (RIS), or another database which acquires and stores medical images, metadata, and/or other electronic health records. The interfaces 102 may include digital imaging and communications in medicine (DICOM), server message block (SMB), common internet file system (CIFS), APPLE filing protocol (AFP), WebDAV, file transfer protocol (FTP), secure file transfer protocol (SFTP), network file system (NFS), hypertext transfer protocol secure (HTTPS), internet protocols (e.g. TCP/IP), secure shell (SSH), transport layer security (TLS), and other communication protocols. The interfaces 102 may be established via hardware/wired connections or wireless connections. The interfaces 102 may connect via one or more APIs to the network databases 101 which may be hosted on cloud servers or as database instances. The databases may be relational databases or other database structures.

The interfaces 102 may transmit one or more image files or other data to a metadata analyzer 104. The computer system 100 or the metadata analyzer 104 may read the metadata of the image data (e.g. DICOM tags) as a preliminary process for the selection and analysis of the image content. This metadata may include, for example, the image modality, image acquisition direction, sequences (for MR modality), slice spacing, but can also contain other data (e.g. features for identifying the patient, image content, acquisition date, etc.). Different medical images, image acquisition devices and language settings result in different metadata for the identical subject matter. In its simplest form, a translation table may stratify the metadata of images of different origin into one consistent terminology, provided by the analysis system. The translation table may be explicitly built by human input but may also be automatically generated by comparing the metadata with actual image content. For instance, an artificial intelligence (AI) program may utilize regression analysis or be trained for natural language processing so as to correlate the image information and metadata and generate a translation table. In order to subsequently process the images, the metadata has to be stored consistent to at least one terminology or similar scheme, allowing programmatic references and processes to analyze the metadata for logic decision triggers.

As an alternative or in addition to capturing or receiving existing metadata (e.g. if data has been removed from the image for data protection reasons) one or more algorithms from the field of machine learning (“AI algorithms”) trained in the classification of image content may be used to extract relevant metadata from the image content (e.g. grey scale pixels or voxels). Such classification metadata derived from the image content may determine in particular the identification and labelling of the anatomical structures visible on the image. The classification metadata may also provide the projection direction in which the image was acquired, and if necessary, which posture the patient has adopted during the image acquisition or in which position (e.g. lying, sitting, or standing) an image was acquired.

The image content for medical images describes the anatomical structures visible in a medical image. For a medical image of a patient's left knee joint, the derived image content metadata may include “left knee joint”, “left tibia”, “left femur”, and/or “left patella”. Or for an anteroposterior (AP) image of the lung, the visible image content may be “lung”, “thoracal spine”, “heart”, and/or “aorta”.

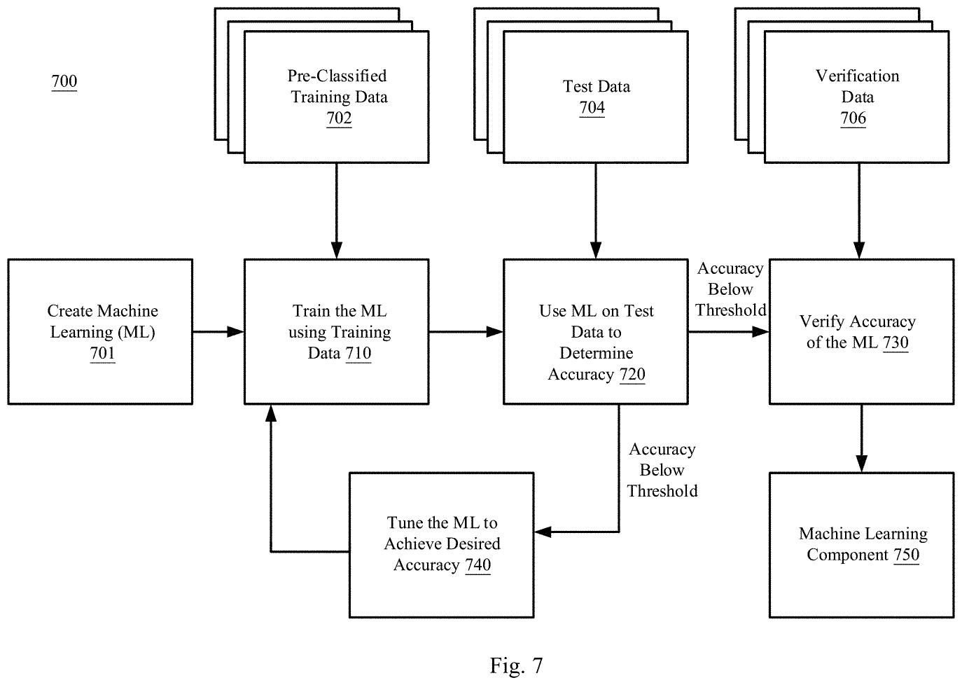

AI algorithms trained as illustrated in FIG. 7 on identifying human anatomy derive the confidence of pixels or voxels belonging to certain structures or organs. If the confidence is above a defined threshold or the difference between the highest ranked structure and the second highest rank is sufficient, the algorithm is capable to derive a segmentation mask indicating the area within the image for the respectively identified structure. The segmentation mask itself or the respective coordinates of bounding boxes or other means allowing to describe the position of the anatomical structures within the image may be saved as input or metadata for subsequent processing. Thus, a reference between a certain area of a visible structure in a medical image and the name or label of said structure is created. For instance, the segmentation mask may relate to image coordinates defining an area.

The metadata can be further enriched with other information by including data, e.g. from the hospital information system, the EHR system, from systems storing laboratory data, imaging data or from other systems. Textual metadata within a medical image, which is not in a structured form can be processed by optical character recognition (OCR) applications and natural language processing (NLP) algorithms to understand the actual content, relationships and thereby derive structured metadata. For example, metal plates X-rayed along with the patient that contain position or patient information may be processed in this manner.

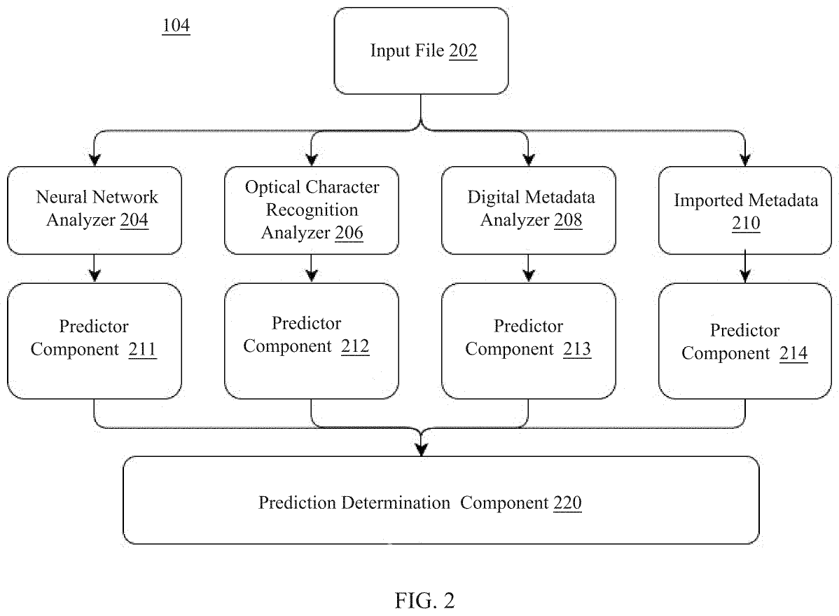

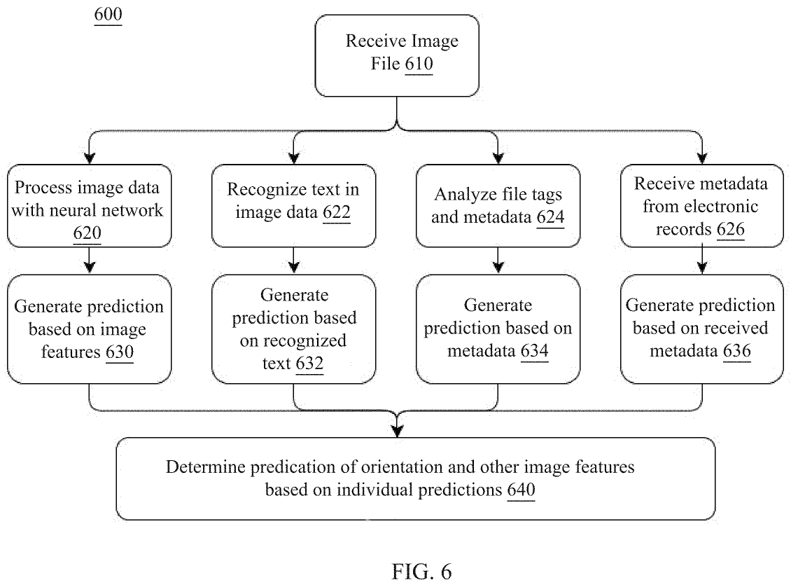

In the metadata analyzer 104 may individually process each of these forms of metadata using separate predictors (prediction engines or classifiers) specialized to the type of input metadata (e.g. NLP for OCR). The metadata analyzer 104 may also process all the metadata of the different origins together and predict the final metadata values (e.g. image orientation, position, posture, imager type, anatomical features, etc.) for the image. The determination of the final metadata may be strictly rule-based, by applying an Al algorithm having generated weighting tables to the various input information (e.g. decision trees, random forest, or state vector machines), or by voting classifiers that rank the prediction confidences of the classifiers operating on the various sources to select a final prediction, or by a combination of such techniques. The final metadata extracted is saved with the image, in a database which has a reference to the image or in other suitable electronic format for subsequent processing. An exemplary structural implementation of the metadata analyzer 104 is illustrated in FIG. 2 and an exemplary process for metadata analysis is illustrated in FIG. 6.

If only certain image data are to be analyzed, one or more filter criteria may be defined. The application of the criteria by the data filter 106 against the available data may take place at this point, so that, for example, only the image data of a certain patient or a group of patients, a certain modality, a certain follow-up period, of certain diseases, for certain treatments or a combination of several of these criteria are subsequently taken into account. A possible special case is the specification of a wildcard filter so that all available image data are included in the subsequent analysis or is passed to the subsequent systems. The data filter can be manually defined by a user via task or filter definition at a user terminal or configured and permanently stored within the system (e.g. in the reference data structures 109). In the stored criteria case, different criteria can be stored for different analyses. The processing of image data by the data filter 106 may be optional if, for example, a database has already been selected and defined to contain the relevant data.

Based on the metadata extracted or aggregated by the metadata analyzer 104, any other data and the filter criteria defined by the data filter 106, the algorithm selector 108 may determine and optimize the image data and algorithms that will used to analyze the respective image content.

In one implementation, optimization criteria (e.g. input requirements) for the algorithms may be stored in a database, configuration file, or in program code that define the suitability of the algorithms for the various applications. For example, a first algorithm may be suitable for segmenting the cervical spine, a second algorithm can be used to segment the lumbar spine, a third to recognize the heads of the femur, a fourth to place landmarks on the sacrum, a fifth to determine the anatomical designation of the segmented vertebrae, a sixth for determining bone density in a given image section, a seventh for classifying degenerative changes, an eighth for determining the extent of carcinogenic tissue, a ninth for counting metastases, etc. If preprocessing by another algorithm is necessary for the application of a subsequent algorithm, this may be identified logically in the program code by corresponding conditions or in the database or configuration file by defining prerequisites of the respective algorithm. The algorithm selector 108 may utilize, manage, or define the database, configuration file or program code to define the suitability. Accordingly, a meaningful sequence of the individual analysis algorithms may thus be determined and an optimized use or application of an algorithm indicated.

The selection of the analysis algorithm or algorithms by the algorithm selector 108 may be made solely on the basis of the metadata of the respective image. If, for example, an image with an artificial hip joint prosthesis is recognized, all algorithms suitable for examining hip joint prosthesis (e.g. cup orientation, wear condition, signs of osteolysis or radiolucency) can be automatically selected for use.

In another implementation, a user (or the system automatically) may specify that all selected or suitable images should be examined for the presence of tumors, for the assessment of the fusion state of the intervertebral disc space, or for constrictions of the vascular system, to name a few examples. The algorithms to be applied can either be explicitly specified by the user or selected based on criteria associated with the analysis task and information related to the capability of each individual algorithm or programmatically based on the derived image metadata.

The algorithms selected or determined for each image by the algorithm selector 108 may then be provided to the computer system 100 and executed to analyze each of the images. The algorithms utilized by the image analyzer 110 may be of different types. For example, deep learning or artificial neural network algorithms (e.g. convolutional neural networks (CNNs), recurrent neural networks (RNNs), long-short term memories (LSTMs)) may segment image areas or place landmarks or markers. Other algorithms may register (“registration algorithms”) the previously segmented image areas through rigid or elastic transformation between different acquisition times. Other algorithms may mathematically describe and determine a surface of segmented 3D data by means of a triangulated surface or active shape models (e.g. mesh models). Other algorithms may calculate or perform the actual transformation of image areas between different images, distances, or angles in 3D or in 2D. For morphological analysis or the definition of access paths (trajectories), algorithms from the field of trigonometry and vector algebra can be used and combined with the previous algorithms, e.g. by connecting landmarks with lines, arcs, splines, analytically determining planes using landmarks and/or a combination of and landmarks, or approximating them using optimization algorithms (e.g. least square, Newton, CMA-ES, LM-CMA, L-BFGS, Bayesian Optimization).

In an implementation, the training data for the machine learning algorithms used for image analysis has been preselected based on additional data so that the prediction quality of the machine learning algorithm may be improved as illustrated in FIG. 6. The additional data may be image data that includes visible surgical interventions in an orthopedic application, e.g. placed implants, components, osteotomies, or resection edges, the application or lack of bone cement, etc. This information may be initially extracted by automated image analysis in the metadata analyzer 104 to extract quantitative values (e.g. minimum/maximum of bone cement, position of the distal and proximal feature of an implant). Such information may serve as ground truth, e.g. for the placement of “interventional landmarks” at the implant features or to correlate parameters of the surgical process with the clinical outcome.

For example, based on “interventional landmarks”, an appropriately trained machine learning algorithm can determine the correct position of an implant or the resection edges on an unknown image. If the training data set consists of image data from an individual surgeon or a small group of surgeons (e.g. within one site), the system can also learn personal preferences and automatically apply them to future images.

In another implementation, the training of the Al algorithms is based on image data sets that are, for example, matched with pathohistological examinations to improve the data quality. By including outcome data in the selection of the training data set, it can also be influenced in such a way that interventions with an above-average treatment outcome form the basis of the machine learning algorithms and thus prognosis or suggestions derived by the algorithm. In addition, historical data with below-average results may be analyzed and correspondingly unfavorable implants, implant sizes, positions/alignments or other parameters related to an intervention may be identified accordingly and deliberately avoided in relation to the patient's surrounding structures or treatment.

In any case, the image analyzer 110 may perform differently based on the algorithms chosen by the algorithm selector 108. The image analyzer 110 may be formed of one or more machine learning models that have been trained on similar data (e.g. local hospital data) or general data as a preliminary step that optimizes their capabilities as noted in FIG. 6. In addition to the results determined by the algorithms, values for probability, confidence intervals for the various outputs of the algorithms, and the minimum/maximum of a cost function can also be calculated and stored for each of the image analysis features. Thus, the image analyzer 110 may also produce results and the values used to assess probability or confidence in the outputs related to the anatomical or pathological structure(s) analyzed.

The computer system 100 may also include referenceable structures and value ranges/analysis parameters (e.g. header files, relational structures, decision trees, etc.). The structures that can be analyzed with the system or the method are stored in at least one database or another data format in such a way that they can be referenced. Geometric references between individual structures are defined in a preferred embodiment, which can include both the basic spatial arrangement and standard values or plausible value ranges for the respective measurements. For example, the order of the vertebra (C1, C2, C3 etc.), the value range of the position (e.g. center C2=center C1 minus 10 . . . 30 mm in Y-direction, +10. . . −10 mm in X-direction) or the value range of angles or distance dimensions, such as the intervertebral disc height between C1 and C2 (0 . . . 9 mm), can be stored. The measurement results the image analyzer 110 are related to the structures and the associated value ranges at least before or during the calculation.

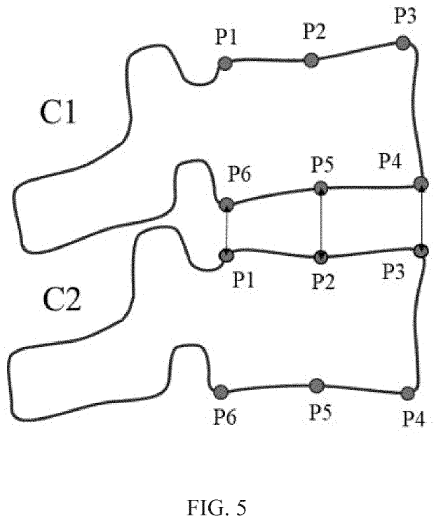

In another implementation, the analysis parameters (e.g. height of the intervertebral disc between C1 and C2) are directly linked or indirectly logically determinable by the structures necessary for the automatic calculation of the height (lines, trajectories, landmarks, image areas, volume areas) by applying characteristic landmarks of a structure (e.g. the corner points of a vertebra) to the respective referenced structures (vertebra C1, vertebra C2) as illustrated in FIG. 5. The resulting connections create a combination between certain landmarks and the structures which are advantageous for the generic, automated calculation. For example, the mean value of the distances (P6_{c1}P1_{c2}, P5_{c1}P2_{c2}, P4_{c1}P3_{c2}) can be calculated to determine the mean disc height between vertebrae C1 and C2.

In a further implementation with the aim of pre-surgical planning of surgical interventions, constraint conditions can be defined which serve to determine the optimal implant position, e.g. minimum screw spacing, maximum deflection angle, minimum wall thickness of the surrounding bone, trajectories to minimize iatrogenic trauma, etc. Subsequent automated simulations, such as the range of motion of a prosthesis before impingement occurs, FEA or reverse kinematics simulations to determine loads and stresses or other simulations, such as blood flow, flow resistance, fatigue etc. may be applied, with a feedback loop to update the planning for optimized parameters or without feedback for documentation purposes.

During or after the algorithms have completed the image analysis, a result validator 112 may evaluate the results of the image analyzers 110 in terms of plausibility, confidence, applicability and/or reliability. This may be done automatically by comparison with stored value ranges in the reference data structures 109, by comparison of values between similar or adjacent structures (e.g. joint gap right/left hip joint, height of intervertebral disc below or above), by correlating data with average data, or by visualization, e.g. in the form of diagrams, in text form, or superimposed on the image data which was the basis of a given the analysis. If a result is not plausible or confident enough, then the result validator may communicate with the algorithm selector 108 to select a new algorithm or adjust certain parameters for the image analyzer 110 in the reference data structures 109. The result validator 112 may then cause the computer system 100 to restart the analysis or perform a parallel analysis beginning again at the algorithm selector 108.

In an implementation, the computer system 100 provides the possibility of making manual corrections via a graphical user interface or a user terminal. Such changes may be permanently stored in the underlying image data and/or a database, along with the updating of the measured values. The computer system 100 or the result validator 112 may also distinguish between measurements with high measurement reliability and those with low measurement reliability by comparing the measured values with the plausible value ranges or by comparing characteristic values of measurement reliability with corresponding limits. Measurements with low measurement reliability could thus be automatically rejected or subjected to a manual check.

A special case of result validation by the result validator is the setting of validation criteria, which classify each calculated result as acceptable, equivalent to the omission of the step of result validation entirely. For example, the algorithms may have been validated in another way.

The data validated by the result validator 112 may be passed to or transmitted to the result generator 114 which may output to a graphical user interface, a printed report, or other aggregation of the image data and results. The result generator 114 may format the results for display (e.g. in XML, HTML, LATEX, PDF, or other document format). The result generator 114 may also transmit the image data and results back to the network database via the interfaces 102 so that the results can be used differently by downstream processes.

For example, the results may be used for research purposes (observing and improving healing processes) by statistically analyzing the extracted and aggregated data The results may be used for the diagnosis and classification of diseases, for the definition of retrospective ad hoc control groups for clinical studies, or for patient-specific support of surgical procedures. For example, by parametrically adapting a computer aided design (CAD) model on the basis of automatic image analysis, the geometry of drilling and cutting guides or patient-specific implants can be defined fully automatically as part of the results.

The results may also include trajectories, lengths and diameters of bone screws can be calculated fully automatically. These results may support the size selection of implants, the documentation of surgical planning, or the procedure itself. Further algorithms can be applied to the results to further calculate orthopedic corrections (e.g. the positioning or size selection of the component of a hip shaft implant or the change in lordosis/kyphosis to create a physiological sagittal profile of the spine). The results obtained may also be used for pre- and/or post-operative documentation, planning of the surgical procedure and calculation of movement paths for robot-assisted surgical procedures. For the latter applications, the automatically generated data is preferably made available via an interface to the RIS/PACS/HIS/EHR database or other suitable systems for storage or further processing.

FIG. 2 illustrates an implementation of the metadata analyzer 104 that provides various components for processing and generating the various pieces of information that determine the final metadata prediction. A file input 202 is an interface or connection to the interfaces 102 which receives one or more image data files and/or one or more metadata files. The image file may then be transmitted to a neural network analyzer 204 for determination of anatomical features, segmentation analysis, or other image-based or neural classifier analysis. The output of the neural network analyzer 204 may then be passed to a specialized predictor component 211 that uses this extracted information to predict the metadata for the image (e.g. posture, direction, position, anatomy). The image file may also be transmitted to an optical recognition analyzer 206 which may operate on the image to identify and extract characters and words from the image. These characters or words may be further analyzed by natural language processor classifiers to extract content, context, and meaning. The output of the optical recognition analyzer 206 may then be transmitted to a predictor component 212 that uses this extracted information to predict the metadata for the image (e.g. posture, direction, position, anatomy).

The image data may also be transmitted to a digital metadata analyzer 208 that may extract metadata sent along with the image in the image file (e.g. headers). The digital metadata analyzer 208 may then output to a specialized predictor component 213 that processes the extracted metadata to predict posture, direction, position, anatomy and other metadata for the image file. The input file may be accompanied by a separate metadata file which may be sent to the imported metadata 210 or this component may access the interfaces 102 to request the associated metadata. This imported metadata may be then transmitted to a predictor component 214 that uses this received information to predict the metadata for the image (e.g. posture, direction, position, anatomy).

The prediction determination component 220 may analyze the different predictions or the different metadata from one or more of the predictors 211, 212, 213 and 214 which produced a result. The predictors 211, 212, 213 and 214 may provide confidence intervals along with their predicted metadata. The prediction determination component 220 may provide a voting classifier, a state vector machine, a decision tree, an optimization table, or other decision process to select the best metadata from one or more of the predictors 211, 212, 213 and 214.

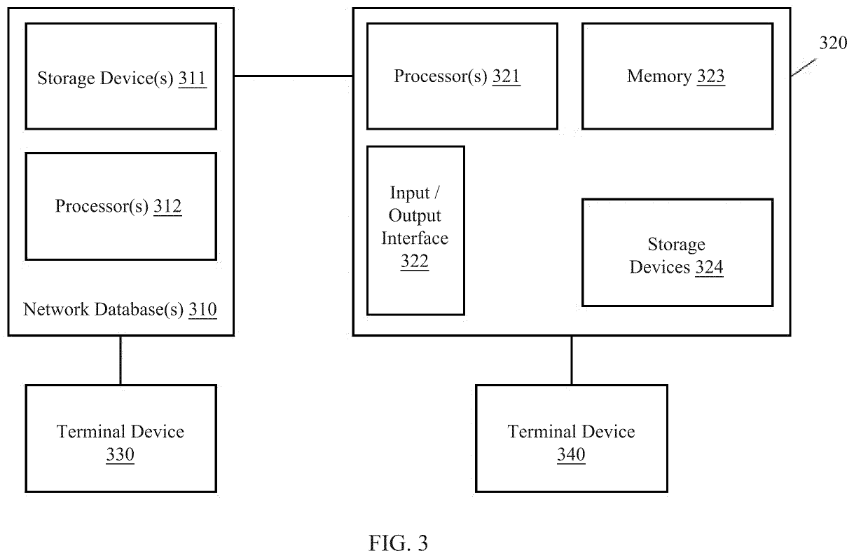

FIG. 3 illustrates the connections to a computer system 320 that allow the computer system 320 (or computer system 100) to interface with users and databases. Specifically, computer system 320 includes one or more processors 321, computer memory 323 (e.g. RAM), an input/output (I/O) interface 322, and one or more storage devices 324. The processors 321 may execute computer instructions to perform the functions of the metadata analyzer 104, the data filter 106, the algorithm selector 108, the image analyzer 110, the result validator 112, and the result generator 114.

The computer system 320 connects to the network databases 310 (e.g. network databases 101) which include storage devices 311 and processors 312 in addition to an interface to connect to the I/O interface 322 of the computer system 320. The connection between the network databases 310 and the computer system 320 may be hardware or software, or a combination such that clinical information stored at the network database 310 may be provided to the computer system 320 securely and efficiently. A terminal device 330 (e.g. a user terminal, desktop computer, or portable device) may connect to the network database 310 to upload image data. Other devices such as medical imaging devices may also connect to the network databases 310.

Likewise, a terminal device 340 connects to the computer system 320 and may be used to input filter data to the data filters 106 or input data to the reference data structures 109. The terminal device 340 may be used to optionally control one or more points of the automated image analysis process. The terminal device 340 may also connect to the result generators 114 to receive the data analysis results and the processed image files for display to the user in a graphical user interface (GUI).

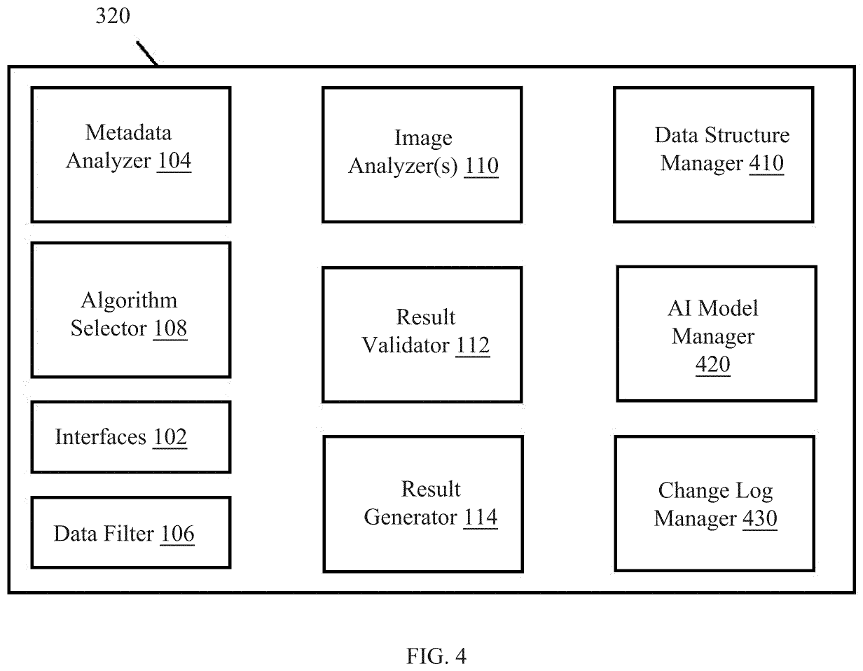

FIG. 4 illustrates a detailed implementation of the computer system 320 or computer system 100. The computer system 320 may include computer applications or components such as the metadata analyzer 104, the data filter 106, the algorithm selector 108, the image analyzer 110, the result validator 112, and the result generator 114. In addition, the computer system may provide or include a computer application comprising computer instructions that manages the data structures used during a single image analysis or a sequence of image processes, the data structure manager 410. The data structure manager 410 may manage, store, or organize the reference data structures 109.

In addition, the computer system may provide or include a computer application comprising computer instructions that manages the various artificial intelligence models and machine learning models of the computer system, the Al model manager 420. In addition, the computer system may provide or include a computer application comprising computer instructions, the change log manager 430, that tracks changes to image files and records the originals along with overlays, markers, measurements, transforms, etc. that may be used to enhance or manipulate the image in the final result. The change log manager 430 may provide flexibility on which image analysis to display in the final result.

FIG. 5 illustrates a final result of an automated image analysis according to an implementation. The automated image analysis recognized that the image was of a segment of a spinal column, and may identify the specific vertebrae. The automated image analysis then generated various markers and analysis parameters as illustrated. The various analysis parameters (e.g. height of the intervertebral disc between C1 and C2) are directly linked or indirectly logically illustrated by the structures necessary for the automatic calculation of the height (lines, trajectories, landmarks, image areas, volume areas) by applying characteristic landmarks of a structure (e.g. the corner points of a vertebra) to the respective referenced structures (vertebra C1, vertebra C2). The resulting connections create a combination between certain landmarks and the structures which are advantageous for the generic, automated calculation. For example, the mean value of the distances (P6_{c1}P1_{c2}, P5_{c1}P2_{c2}, P4_{c1}P3_{c2}) can be calculated to determine the mean disc height between vertebrae C1 and C2.

The schematic flowchart illustrated in FIG. 6 provides a process of predicting final metadata information about a medical image. For metadata prediction of the metadata analyzer 104, at least one source of information is used, (e.g. existing metadata from the medical image). Additional metadata, such as that derived by directly analyzing the image content, textual content that may be available within the image, or information that maybe accessible through related information systems with reference to the patient or medical image are incorporated before a final prediction of the metadata is created in a terminology suitable for further processing. The metadata subsequently serves as trigger for the selection of suitable analysis algorithms at the algorithm selector 108.

The various sources of metadata may each have separate process for extraction and generation or predictions. One or more of the processes may be used depending on the availability of the source information (e.g. the image file may contain no text to recognize). At 610, the metadata analyzer 104 may receive an image file. At 620, the metadata analyzer 104 may process the image data with one or more neural network classifiers to identify image or voxel content. At 630, the image or voxel content may be processed by a specialized predictor to generate a prediction based on the image features.

At 622, the metadata analyzer 104 may recognize text in the image data using OCR and NLP algorithms. At 632, the metadata analyzer 104 may generate a prediction of metadata based on the recognized text and its context. At 624, the metadata analyzer 104 may analyze or parse the image file tags and associated metadata such as header data (e.g. DICOM tags). At 634, the metadata analyzer 104 may generate a prediction of metadata based on the parsed metadata (e.g. add details typically associated with the provided metadata). At 626, the metadata analyzer 104 may receive metadata from one or more electronic data systems (e.g. RIS, LAB, EHR data). At 636, the metadata analyzer 104 may generate one or more predictions or filter the received data to arrive at the relevant metadata for the image file. The generated predictions from the processes at 630, 632, 634, and 636 may feed the predictions into a final process 640 that determines prediction of orientation and other image features based on the individual predictions. The final process 640 may operate a voting classifier or other selection method to take the best data from each of the separate sources. The individually illustrated processes 630, 632, 634, 636, and 640 may operate together as a single process taking in the separate sources of processed data from processes 620, 622, 624, and 626.

FIG. 7 is a flowchart of a process 700 to train a machine learning algorithm, according to some implementations. The process 700 may be performed by the computer system 100 or 320.

At 701, the machine learning algorithm (e.g., software code) may be created by one or more software designers. At 710, the machine learning algorithm may be trained using pre-classified training data 702. For example, the training data 702 may have been pre-classified by humans, by machine learning, or a combination of both. After the machine learning has been trained using the pre-classified training data 1702, the machine learning may be tested, at 720, using test data 704 to determine an accuracy of the machine learning. For example, in the case of a classifier (e.g., support vector machine), the accuracy of the classification may be determined using the test data 704.

If an accuracy of the machine learning does not satisfy a desired accuracy threshold (e.g., 95%, 98%, 99% accurate), then at 740, the machine learning code may be tuned, to achieve the desired accuracy. For example, at 740, the software designers may modify the machine learning software code to improve the accuracy of the machine learning algorithm or prune the training data. After the machine learning has been tuned, at 740, the machine learning may be retrained, at 710, using the pre-classified training data 702. In this way, 710, 720, and 740 may be repeated until the machine learning is able to classify the test data 704 with the desired accuracy.

After determining that an accuracy of the machine learning satisfies the desired accuracy threshold, the process may proceed to 730 where verification data for 706 may be used to verify an accuracy of the machine learning. After the accuracy of the machine learning is verified, at 730, the machine learning component or logic 750, which has been trained to provide a particular level of accuracy, may be used. The process 1700 may be used to train each of multiple machine learning algorithms. For example, as part of the metadata analyzer 104 or the image analyzer 110, a first machine learning may be trained to make first predictions, a second machine learning may be trained to predict second predictions, and a third machine learning may be trained to predict third predictions, and so on. These various machine learning models may then be selected by the algorithm selector 108 based on their optimizations and training. The trained model may encompass specialized computer instructions, reference data structures, and/or one or more layers or connections between a data input and a data output.

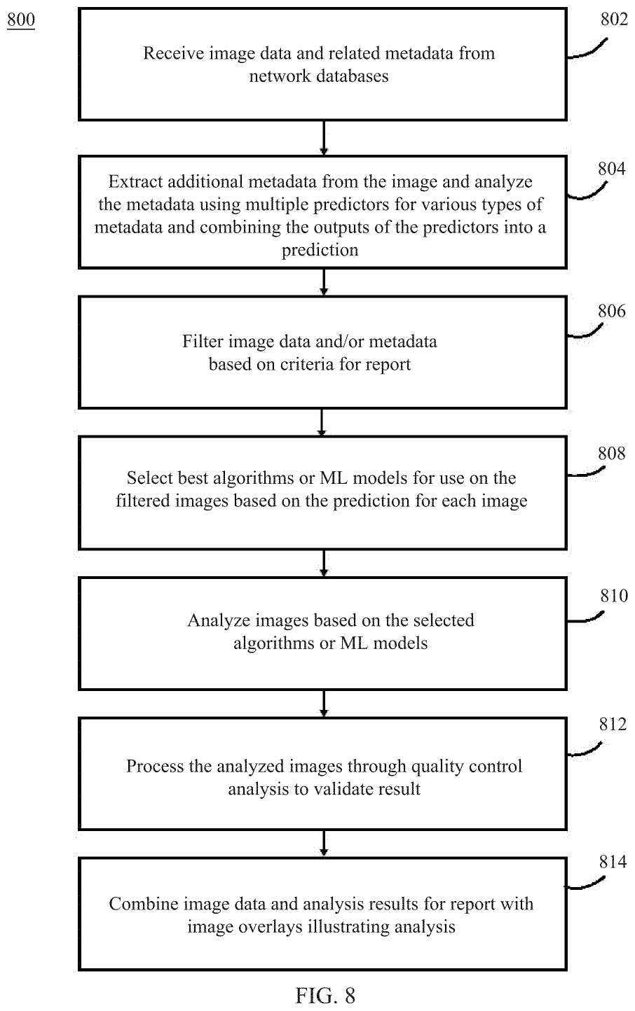

FIG. 8 illustrates an automated image processing process 800 for clinical image data. At 802, the computing system 320 or process 800 receives image data and related metadata from network databases. At 804, the computing system 320 or process 800 may extract additional metadata from the image and analyze the metadata using multiple predictors for various types of metadata and combine the outputs of the predictors into a prediction of the metadata for the image file (e.g. pose, position, orientation, direction, posture, anatomical feature, etc.). At 806, the computing system 320 or process 800 may optionally filter the image data and/or metadata based on criteria for the report or input by the user. At 808, the computing system 320 or process 800 may select the best algorithms or ML models for use on the filtered images based on the prediction of metadata for each image.

At 810, the computing system 320 or process 800 analyzes the images based on the selected algorithms or ML models (e.g. CNN, RNN, LTSM) and outputs an analysis result along with a confidence interval. At 812, the computing system 320 or process 800 may process the analyzed images through quality control analysis to validate result based on confidence thresholds or the like. At 814, the computing system 320 or process 800 may combine the image data and analysis results for a report with image overlays illustrating analysis, or other image markup or data display (e.g. deviations from average anatomical sizes).

The structures and parameters that can be analyzed are stored in a database or similar data format as reference data structures. The combination of structure and parameters can also contain permissible value ranges, which can then be used for image analysis, result validation or result usage.

Furthermore, this disclosure provides various example implementations, as described and as illustrated in the drawings. However, this disclosure is not limited to the implementations described and illustrated herein, but can extend to other implementations, as would be known or as would become known to those skilled in the art. Reference in the specification to “one implementation,” “this implementation,” “these implementations” or “some implementations” means that a particular feature, structure, or characteristic described is included in at least one implementation, and the appearances of these phrases in various places in the specification are not necessarily all referring to the same implementation.

Although the present invention has been described in connection with several embodiments, the invention is not intended to be limited to the specific forms set forth herein. On the contrary, it is intended to cover such alternatives, modifications, and equivalents as can be reasonably included within the scope of the invention as defined by the appended claims.

What is claimed is:

1. A method for the automatic extraction and processing of data from medical images by computer instructions executed on one or more processors and at least one interface to an image data archive, comprising:
generating a metadata for an image;
selecting an algorithm for image data analysis based on the metadata generated for the image, properties for at least two possible algorithms, and a specification of a specific image analysis to be performed;
analyzing the image data with the algorithm selected from the at least two possible algorithms to produce results information;
linking the results information of image analysis and the metadata with referenceable anatomical structures of a human being or an animal within the image; and
displaying the results linked to the anatomical structure.

2. The method of claim 1, further comprising: filtering the image by matching the metadata against filter criteria.

3. The method of claim 1, further comprising: evaluating the results information by comparison with value ranges or thresholds indicative of the confidence of the correctness.

4. The method of claim 1, wherein the medical images are enriched with embedded metadata derived by analysis of contents of the medical images with the aid of artificial intelligence algorithms.

5. The method of claim 1, wherein the medical images are enriched with metadata from other data sources.

6. The method of claim 1, wherein, in generating the metadata, image-based textual data is analyzed and standardized through optical character recognition and natural language processing algorithms and output as the metadata.

6. The method of claim 1, wherein, in generating the metadata, different sources of extracted metadata are jointly analyzed for a final prediction output as the metadata, wherein the metadata is structured in a predefined terminology for subsequent processing.

7. The method of claim 1, wherein, in the selecting of the algorithm for image analysis, the selection is based on a stored association between an analysis task and the algorithm.

8. The method of claim 1, wherein, in the selecting of the algorithm for image analysis, the selection is based on a stored association between an analysis task and the metadata of the image.

9. The method of claim 1, wherein the at least two possible algorithms include: AI algorithms, computer vision, segmentation, registration, trigonometry, vector algebra, optimization functions, and/or digitally reconstructed radiographic image projection (DRR).

10. The method of claim 1, wherein the results information of the algorithm are associated with a metric suitable for assessing a correctness of the results information.

11. The method of claim 1, wherein the results information are referenced to structures of an animal or human being within the image.

12. The method of claim 1, wherein a permissible range of values for the results information is limited.

13. A system for an automated analysis of medical images, comprising:
a computer including memory, a processor, and an external interface suitable to establish a connection to at least one external computer system for exchanging medical image data,
wherein the computer includes program instructions, loaded through the external interface or from storage media of the computer, that when executed implement the method according to claim 1.

14. A computer readable medium including computer readable instructions that when executed perform the functions of claim 1.
